(19) United States
(12) Patent Application Publication
(10) Pub. No.: US 2017/0319744 A1
(43) Pub. Date: Nov. 9, 2017

(54) COMPOSITE POLYMERIC NANOFIBERS FOR SKIN REGENERATION

(71) Applicant: Mohammad RAOUFI, Tehran (IR)

(72) Inventor: Mohammad RAOUFI, Tehran (IR)

(21) Appl. No.: 15/658,364

(22) Filed: Jul. 24, 2017

Related U.S. Application Data
(63) Continuation-in-part of application No. 15/516,396, filed on Apr. 1, 2017.

(51) Int. Cl. A61L 27/26; A61L 27/38; A61L 27/42; A61L 27/56; D01F 4/00; D01D 5/00; B29C 47/00; A61L 27/60; D01F 9/00; A61F 2/10; B29L 31/00

(52) U.S. Cl. CPC A61L 27/26; D01F 9/00; A61L 27/3804; A61L 27/3813; A61L 27/427; A61L 27/56; A61F 2/105; D01F 4/00; D01D 5/00; B29C 47/0014; B29C 47/0004; A61L 27/60; B29L 2031/7532; A61F 2240/001; A61L 2400/12; B29K 2089/00; D10B 2509/00

(57) ABSTRACT

A method for preparing a skin regeneration scaffold is disclosed. The method may include preparing a polymer solution by dissolving a biopolymer in a solvent, and subjecting the polymer solution to a template-assisted extrusion process with a nanoporous material as a template in order to produce polymer nanofibers. Furthermore, the method includes fabricating a multilayer composite nanofibrous scaffold using the polymer nanofibers. The composite nanofibrous scaffold may be seeded with cells. In some cases, the cells may be selected from autologous cells, allogeneic cells, or combinations thereof.

### CROSS-REFERENCE TO RELATED APPLICATION

This application is a continuation-in-part of U.S. patent application Ser. No. 15/516,396, filed on Apr. 1, 2017, and entitled “METHODS FOR PREPARING AND ORIENTATING BIOPOLYMER NANOFIBRES AND A COMPOSITE MATERIAL COMPRISING THE SAME,” which is a National Stage of International Patent Application PCT/EP2015/001942, filed on Oct. 2, 2015, and entitled “METHODS FOR PREPARING AND ORIENTATING BIOPOLYMER NANOFIBRES AND A COMPOSITE MATERIAL COMPRISING THE SAME,” which claims priority to European Patent Application Number EP 14003414,1, filed on Oct. 2, 2014, The disclosures all of the foregoing applications are incorporated by reference herein in their entirety.

### TECHNICAL FIELD

The present application generally relates to the field of skin regeneration and the preparation of skin substitutes, and more particularly to methods and formulations for the preparation of skin substitute scaffolds for various wound types.

### BACKGROUND

Accidents, trauma, and burns can often cause skin damage to subjects over a large area. In addition, conditions such as chronic vascular diseases and diabetes, as well as aging and pressure ulcerations due to long-term hospitalizations, can result in non-healing or slow-healing wounds that have presented significant clinical challenges. For example, recent data published by the Center for Disease Control and Prevention has shown that more than 20 million individuals have developed diabetes. Furthermore, over 2 million of these individuals were diagnosed with chronic diabetic ulcers. Unfortunately, more than 5% of chronic diabetic ulcer cases eventually lead to amputation.

Currently, the most common clinical treatments for skin transplantation include split-thickness, full thickness, or composite grafts. However, current skin grafts have their own shortcomings. For example, it can often be difficult to obtain skin grafts from patients with chronic diseases, as such persons are unable to endure large-scale operations and anesthesia. Moreover, as skin grafts lack a higher expansion ratio, itchy and painful hypertrophic scar tissue may form. Furthermore, this process is relatively expensive, labor intensive, and complex to implement.

As an alternative to skin grafts, many engineered skin substitutes have been developed and used clinically. These skin substitutes employ the concept of tissue engineering, combining biomaterials, cells, and/or growth factors to accelerate the regeneration process. For example, products such as Laserskin, CellSpray, and BioSeed-S have been used as epidermis substitutes.

Other products such as Integra. AlloDerm, and Biobrane have been used as cellular dermis substitutes, and products such as Derma graft have been used as cellular dermis. These bioengineered products contain scaffolds which, depending on the application, may require cell harvesting followed by cell seeding on the scaffolds. Despite some effectiveness, these products are lab-intensive, complex, and require costly procedures to implement. Moreover, the successful use of such bioengineered products are typically related to the underlying disease causing the skin condition. For example, for diabetic type wounds, such scaffolds must support fast cell growth, empower the immune system, and remove the risk of bacterial infection.

Therefore, there is a need in the art for a scaffold that meets these requirements and can aid in the repair of damaged or diseased skin tissue. There is further a need in the art to develop a scaffold that is relatively inexpensive while exhibiting good mechanical strength, beneficial biological properties, and facilitate cell development and metabolism.

### SUMMARY

This summary is intended to provide an overview of the subject matter of this patent, and is not intended to identify essential elements or key elements of the subject matter, nor is it intended to be used to determine the scope of the claimed implementations. The proper scope of this patent may be ascertained from the claims set forth below in view of the detailed description below and the drawings.

In one general aspect, the present disclosure describes a method for preparing a skin regeneration scaffold. The method may include one or more of the following steps: providing a nanoporous material, preparing a polymer solution by dissolving a biopolymer in a solvent, subjecting the polymer solution to a template-assisted extrusion process with the nanoporous material as the template in order to produce polymer nanofibers, and fabricating a multilayer composite nanofibrous scaffold using the polymer nanofibers.

The above general aspect may include one or more of the following features. The method for preparing the skin regeneration scaffold may further include a step of separating the polymer nanofibers from, the solvent, and/or subjecting the multilayer composite nanofibrous scaffold to a plastic compression. In some cases, the method also includes seeding the composite nanofibrous scaffold with cells, where the cells are selected from the group consisting of autologous cells, allogeneic cells, and combinations thereof. In another implementation, the cells can be selected from the group consisting of keratinocyte, fibroblasts, and combinations thereof. In one implementation, the cells are selected from the group consisting of keratinocytes, fibroblasts, melanocytes, endothelial cells, chondrocytes, osteocytes, osteoblasts, stem cells, and bone marrow.

In addition, in some cases, the seeding the composite nanofibrous scaffold with the cells can further include growing a layer of a first type of cell on a first side of the scaffold and growing a second type of cell on a second side of the scaffold. In some cases, the first type of cell may include keratinocytes, while in other cases, the first type of cell can include fibroblasts. In another example, the first type of cell and the second type of cell can be selected from the group consisting of keratinocytes, fibroblasts, melanocytes, endothelial cells, chondrocytes, osteocytes, osteoblasts, stem cells, and bone marrow.

According to some implementations, the nanoporous material may be selected from anodic aluminum oxide (AAO), titanium dioxide, silicon dioxide, polycarbonate, or a zeolite. Furthermore, the nanoporous material may have a mean pore size in a range of 4 nm to 900 nm. According to another implementation, the nanoporous material may have a thickness in a range of 10 μm to 400 μm. In another example, the nanoporous material may be an AAO membrane with a mean pore size in a range of 10 nm to 150 nm.

Furthermore, in some implementations, the biopolymer may be selected from proteins, polysaccharide, or combinations thereof. In some cases, the biopolymer may be selected from fibronectin, elastin, fibrinogen, collagen, myosin, actin, BSA, α-actinin, laminin, chondroitin sulfate, hyaluronan, chitin-derivatives, or mixtures thereof.

According to one implementation, the template-assisted extrusion process may include extruding the polymer solution through pores of the nanoporous material, where extruding the polymer solution through pores of the nanoporous material may be carried out by either pressing the polymer solution through the pores of the nanoporous material or drawing the polymer solution through the pores of the nanoporous material. As another example, the step of fabricating the multilayer composite nanofibrous scaffold using the polymer nanofibers may further include depositing the polymer nanofibers by a layer-by-layer approach on a substrate. In another implementation, the method for preparing the skin regeneration scaffold may further include a step of applying mechanical pressure or cross-linking the multilayer composite nanofibrous scaffold by a freezing-thawing method. The application of mechanical pressure may be carried out by a plastic compressor, which applies a suitable pressure at a specific temperature. The freezing thawing method may include freezing the scaffold at −20° C. and thawing the scaffold to the room temperature.

According to another general aspect, the present disclosure describes a skin regeneration scaffold prepared by the methods detailed herein. In another general aspect, the present disclosure describes a skin substitute or artificial skin that may include a multilayer composite nanofibrous scaffold seeded with keratinocytes and fibroblasts cells.

Other systems, methods, features and advantages of the implementations will be, or will become, apparent to one of ordinary skill in the art upon examination of the following figures and detailed description. It is intended that all such additional systems, methods, features and advantages be included within this description, and this summary, be within the scope of the implementations, and be protected by the following claims.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates an implementation of a preparation method for a skin regeneration scaffold;

FIG. 2 illustrates a sectional view of an implementation of an extrusion device;

FIG. 3A depicts scanning electron microscope (SEM) images of an implementation of AAO membranes with pore diameters of 55 nm from a top view;

FIG. 3B depicts scanning electron microscope (SEM) images of an implementation of AAO membranes with pore diameters of 140 nm from a top view;

FIG. 3C illustrates a cross-sectional view of an implementation of a single pore in an AAO membrane;

FIG. 4 depicts SEM images of an implementation of produced nanofibrous scaffolds; and

FIG. 5 depicts SEM images of an implementation of cultured cells on the composite nanofibrous scaffold.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

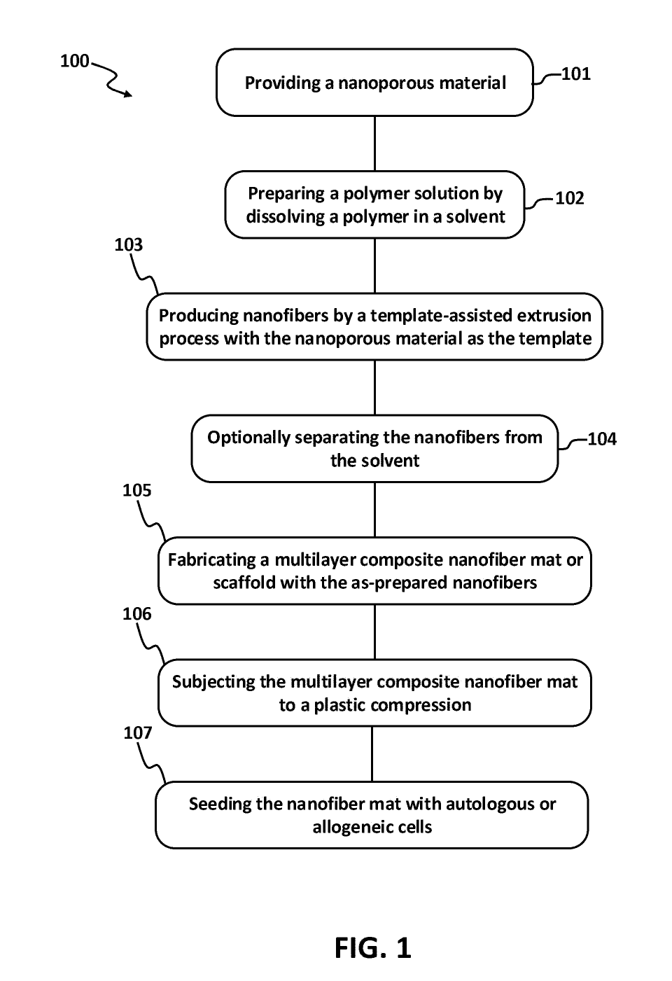

Systems and methods for fabricating composite nanofibrous mats from biopolymers with a template-assisted extrusion method, as well a skin substitute fabricated based on the composite nanofibrous mats and methods for preparation thereof, are disclosed. FIG. 1 illustrates an implementation of a preparation method 100 for a skin regeneration scaffold according to one or more aspects of the present disclosure. As provided herein, in some implementations the method 100 may include a first step 101 of providing a nanoporous material that may be, for example, in a form of a nanoporous membrane or a nanoporous mesh. In addition, the method 100 includes a second step 102 of preparing a polymer solution that may be, for example, carried out by dissolving a polymer in a suitable solvent with a specific concentration. A third step 103 can include producing nanofibers by a template-assisted extrusion process that may be, for example, carried out by pressing or drawing the polymer solution through the pores of the nanoporous material in order to form nanofibers. The method 100 can further include an optional fourth step 104 of separating the nanofibers from the solvent. Furthermore, the method includes a fifth step 105 of fabricating a multilayer composite nanofiber mat (i.e., scaffold) by a layer-by-layer deposition approach, a sixth step 106 of subjecting the scaffold to a plastic compression that may be carried out by applying pressure on the scaffold for a predetermined amount of time, and a seventh step 107 of seeding the composite nanofiber mat with autologous or allogeneic cells. Additional details regarding the method 100 are provided below.

Referring to FIG. 1, in some implementations, the first step 101 may involve providing or forming a nanoporous material, such as anodic aluminum oxide (AAO), titanium dioxide, silicon dioxide, polycarbonate, and/or a zeolite. According to some implementations, the nanoporous material may have a mean pore size in a range of about 4 nm to about 900 nm. In other implementations, the nanoporous material may have a mean pore size in a range of about 20 nm to about 200 nm. Furthermore, in some implementations, the nanoporous material may have a thickness in the range of about 10 μm to about 400 μm. In other implementations, the nanoporous material may have a thickness in the range of about 50 μm to about 200 μm.

In one implementation, nanoporous AAO membranes with pore diameters from 10 to 150 nm may be prepared by an anodization method described in Raoufi et al., “Pushing the Size Limits in the Replication of Nanopores in Anodized Aluminum Oxide via the Layer-by-Layer Deposition of Polyelectrolytes,” Langmuir, 2012, 28 (26), Pages 10091-10096, which is incorporated herein by reference in its entirety and hereinafter referred to as the “Replication of Nanopores” reference, and has been submitted herewith in the present application. In some implementations, the pores may extend substantially the entire thickness of the membrane, with both ends of the pore being open.

In some implementations of second step 102, a natural or synthetic biopolymer, such as for example a protein or a polysaccharide, may be dissolved in a suitable physiological or non-physiological organic or inorganic solvent in order to prepare a polymer solution. For purposes of this disclosure, a protein may refer to any sequence of more than about 10 amino acids, and more specifically a sequence of about 10 to 1000 amino acids. In addition, for purposes of this disclosure, a polysaccharide may encompass any sequence of more than about 10 monosaccharides, and more specifically a sequence of 10 to 1000 monosaccharides that may be different or identical.

According to some implementations, the monosaccharide basic units may include between 3 and 9 carbon atoms, or alternatively, between 5 and 7 carbon atoms. The monosaccharides units may include, for example, glucose, galactose, glucosamine, glucuronic acid, galacturonic acid, acetyl glucosamine, arabinose, fructose, fucose, mannose, rhamnose, sialic acid, and/or derivatives thereof. Furthermore, in some implementations, the biopolymer may include, for example, fibronectin, elastin, fibrinogen, collagen, myosin, actin. BSA, α-actinin, laminin, chondroitin sulfate, hyaluronan, chitin-derivatives (e.g., chitosan), and/or mixtures thereof.

In some implementations, the solvent may include acetic acid or ionic liquids that may be used for polysaccharides, or physiological buffers that may be used for proteins. In addition, other suitable solvents for a specific polymer known in the art may be utilized.

With respect to third step 103, in some implementations, a template-assisted extrusion process may be utilized for producing nanofibers. In the template-assisted extrusion process, the polymer solution may be extruded through the pores of the nanoporous material by either pressing or drawing the polymer solution through the pores of the nanoporous material in order to form the nanofibers. In one implementation, the polymer solution may be pressed or drawn through the nanoporous material, such as for example an AAO porous membrane, using controlled speed and pressure to form polymeric nanofibers.

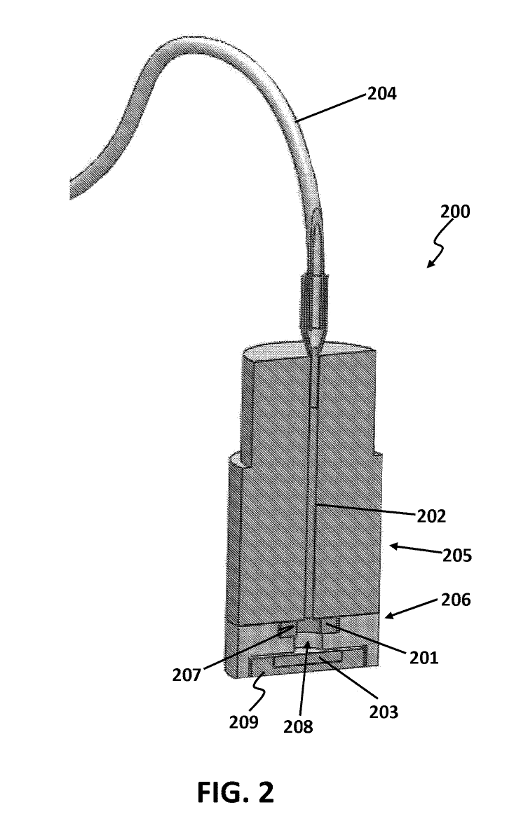

For purposes of clarity, a sectional view of an example of an extrusion device 200 is depicted in FIG. 2. The extrusion device 200 may include a template mount 201 for holding, storing, securing, or otherwise supporting the nanoporous material (i.e., the template) disposed immediately or directly below a channel 202. The channel 202 is configured to guide a pumped polymer solution onto the nanoporous material. The extrusion device 200 also includes a substrate 203 that may be mounted or disposed below the mount 201. The polymer solution may be pumped into the extrusion device 200 via a polymer solution line 204. In some implementations, the polymer solution may be pressed onto the nanoporous material, thereby being extruded through the pores of the nanoporous material in the form of extruded nanofibers. The extruded nanofibers may be collected on the substrate 203. In one implementation, a cleaned glass substrate may be utilized for collecting the extruded nanofibers.

With further reference to FIG. 2, according to some implementations, the extrusion device 200 may include two caps, referred to herein as an upper cap 205 and a lower cap 206. The upper cap 205 may be tightly positioned or secured along a top or upper surface of the lower cap 206 by various fastening means, such as steel screws, clamps, vises, nuts, washers, adhesives, and other fastening systems. The upper cap 205 and, the lower cap 206 may be tightly sealed by the use of for example, O-rings disposed between the upper cap 205 and the lower cap 206, or other sealing agents including but not limited to seals, gaskets, hydraulic seals, hydraulic seals, metric seals, viton, teflon, silicone, rubber, nitrile, and/or plastic or other seals.

In some implementations, the channel 202 may be formed within the upper cap 205 and may be in fluid communication with the line 204. The lower cap 206 can include a housing 207 formed within the lower cap 206 that is configured to facilitate the tight and/or secure placement of the template mount 201 therein. In one implementation, the template mount 201 may be structured as a ring and the nanoporous material may be disposed or mounted inside the ring 201. Furthermore, the lower cap 206 can include a passage 208 formed inside the lower cap 206 below the housing 201. The passage 208 leads downward to a substrate holding section 209 that may be integrally formed with the lower cap 206 or may alternatively be attached, connected, or joined and sealed under the lower cap 206. In addition, the substrate 203 may be tightly disposed or positioned within a recess formed on the substrate holding section 209.

Referring back to FIG. 1, after producing nanofibers by the template-assisted extrusion process in the third step 103, one implementation of which was described in detail above, the nanofibers may optionally be separated from the solvent in a fourth step 104. The separation can occur by, for example, evaporation, centrifugation, sedimentation, or other separation techniques. Moreover, in one implementation, the nanofibers may further be functionalized or purified.

In some implementations, the nanofibers that may be used for preparing composite materials may have a length in the range of, for example, 100 nm to 10 μm, or alternatively they may have a length of, for example, 1 μm to 5 μm. Furthermore, the nanofibers can have a diameter ranging between 10 nm and 140 nm in some implementations.

As shown in FIG. 1, the method can further include fabricating a multilayer composite nanofibrous mat or scaffold by a layer-by-layer deposition in a fifth step 105. This approach may facilitate the fabrication of different nanofiber mats with different porosities. According to some implementations, the composite nanofibrous scaffolds may be applying mechanical properties according to plastic compressor method or cross-linked with a freezing-thawing method. In one implementation, the nanofibers may be frozen at −20° C. for 3 hours and thawed to room temperature for 3 hours. The freeze-thaw cycle may be repeated 3 times.

The fifth step 105 can then be followed by the sixth step 106 of subjecting the scaffold to a plastic compression that may be carried out by applying pressure on the scaffold for a predetermined amount of time. Thus, in some implementations, the method includes applying mechanical pressure on the multilayer composite nanofibrous scaffold by use of a plastic compressor. The plastic compressor allows the application of pressure to occur at a specified temperature and pressure level.

Referring next to seventh step 107, the composite nanofiber scaffold may be seeded with autologous or allogeneic cells, for example, keratinocyte or fibroblasts, in order to obtain a composite polymeric nanofiber scaffold. In one implementation, these cells may be grown on the nanofiber scaffold without any growth factors. The cells may include, for example, keratinocytes, fibroblasts, melanocytes, endothelial cells, chondrocytes, osteocytes, osteoblasts and stem cells originated from the cord blood, bone marrow, adipose tissue and the cells that may be normal, genetically modified, or malignant.

In some implementations, the cells may be grown on one side of the scaffold, or alternatively, the cells may be grown on both sides of the scaffold. According to another implementation, one type of cell may be grown on one side of the scaffold and another type of cell may be grown on the other side of the scaffold.

In some implementations, keratinocyte and fibroblasts may be seeded, with a number of cells on the surface of nanofibrous scaffolds. In one implementation, the number of cells is about 5×10⁴. As an example, keratinocyte cells may be grown on one side of the scaffolds and fibroblasts may be grown on the other side of the scaffolds to obtain composite polymeric nanofibrous scaffolds that may act as an artificial skin. In one implementation, the artificial skin may include a composite nanofibrous scaffold or membrane with a layer of keratinocytes grown on one side of the membrane and a layer of fibroblasts grown on the other side of the composite membrane. In other implementations, the composite nanofibrous scaffold or membrane can include a layer of other autologous or allogeneic cells on each side, as noted above.

In different implementations, the as-produced composite polymeric nanofibrous scaffolds may be utilized for skin regeneration purposes. Furthermore, the composite scaffold of the present disclosure may be utilized as a wound dressing or skin substitute to promote wound healing and/or tissue regeneration. In particular, the composite scaffold can be useful for treating skin damage that may include or result from diabetic ulcers, injuries, dermatological conditions, and other skin diseases or disorders.

### EXAMPLE 1

### Preparation of Scaffolds

In this example, an implementation of the method 100 of FIG. 1 is described for preparing composite nanofibrous scaffolds according to one or more aspects of the present disclosure. In Example 1, AAO membranes are utilized as templates in a template-assisted extrusion process for preparing nanofibers from several different proteins and polysaccharides.

### Nanoporous AAO Membranes

According to one implementation, nanoporous AAO membranes with pore diameters from 18 to 300 nm were prepared by an anodization method described in the Replication of Nanopores reference. In order to prepare AAO membranes with pores that extend the entire thickness or substantially the entire thickness of the membranes, providing a type of through-channel, the underlying aluminum substrate was removed in a solution containing 3.5 g of CuCl₂.H₂O, 100 mL of HCl (37 wt %), and 100 mL of H₂O followed by chemical etching of the nanopores bottom with a 0.5 M aqueous phosphoric acid solution at 35° C.

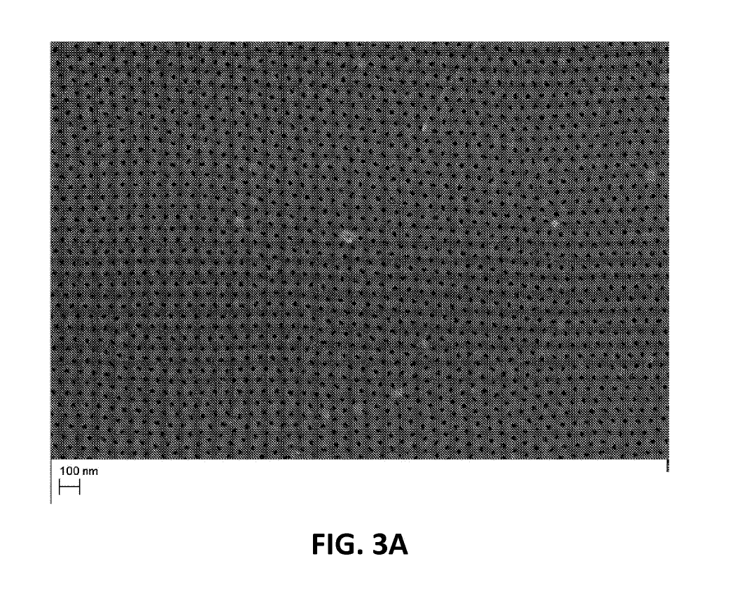

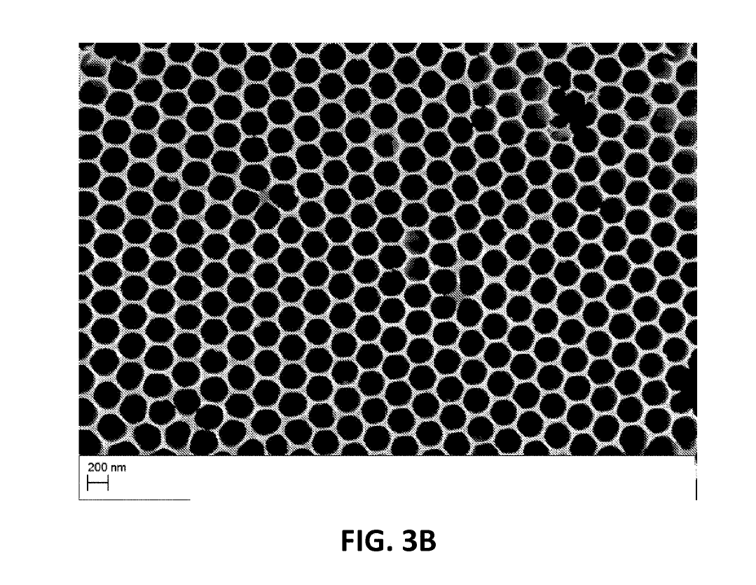

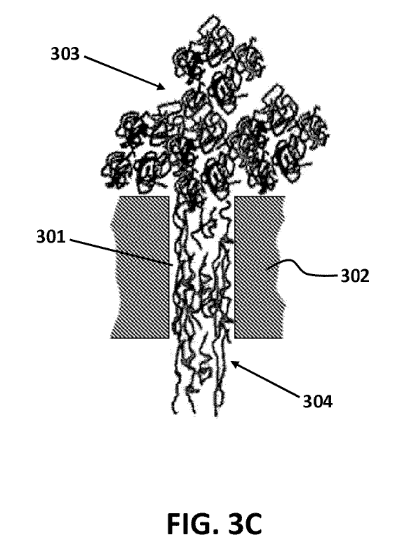

FIGS. 3A and 3B illustrate scanning electron microscope (SEM) images of AAO membranes from top views showing the nanopores of the AAO membrane. In FIG. 3A the pore diameters of approximately 55 nm are depicted, while pore diameters of approximately 140 nm are illustrated in FIG. 3B. Furthermore, the pores extend through the thickness of the AAO membranes and are open at both ends to allow for the polymer solution to be pressed or drawn through these pores and form nanofibers.

### Template-Assisted Extrusion

In order to prepare the composite polymeric nanofibrous scaffolds, as described in connection with FIG. 2, polymer solutions were prepared in different buffers and concentrations according to Table 1 (see below). The polymer solutions were then used as the extrusion feed. It can be understood that the preparation made use of an extrusion device 200. Referring to FIG. 2, polymer solutions were pumped through the line 204 as the extrusion feed into the extrusion device 200. The AAO membrane was mounted on the template mount 201 below channel 202. The upper cap 205 was then tightly fastened on the lower cap 206 and the caps were sealed with two different types of O-rings. A cleaned and dried cultured multiwell plate was used as the substrate 203 and placed inside the substrate holding section 208 under the AAO membrane to collect the extruded composite polymeric nanofibers. The polymer solution was pumped through channel 202 and was electromechanically extruded through the AAO membrane with a rate of 500 μl/min at a substantially constant pressure. The extrusion was collected on the substrate 203.

In different implementations, the polymer solution may include chitosan, elastin, collagen, hyaluronic acid and chondroitin sulphate. In one implementation, the weight ratio of chitosan may be more than 50 wt %. In another implementation, the polymer solution may include chitosan, collagen and chondroitin sulphate. In yet another implementation, the polymer solution may include chitosan, collagen, elastin and hyaluronic in which the weight ratio of chitosan may be greater than or equal to 80%.

FIG. 3C illustrates a cross-sectional view of a single pore 301 in the AAO membrane 302. Referring to FIG. 3C, pore 301 can be understood to extend through the entire thickness of the AAO membrane 302 and it may be open at both ends. The polymer solution 303 may be pressed or drawn through the pore 301 to form nanofibers 304. The nanofiber 304 may later be collected, layer by layer, on a substrate to form a polymeric composite nanofibrous scaffold.

In the present example, the produced polymeric composite nanofibrous scaffold was then dried at room temperature under a clean hood for 30 min. The scaffold was subsequently get under plastic compressor method or cross-linked with a freezing-thawing method. The freezing-thawing method involved freezing the composite polymeric nanofibers at −20° C. for 3 hours and then thawing the nanofibers to room temperature, maintaining the nanofibers at room temperature for 3 hours. This freeze-thaw cycle was repeated 3 times. The plastic c compressor method may include the applying the suitable force (depends on size of scaffold) on special temperature.

TABLE 1
Composition of the polymer solution used as the feed in the template-
assisted extrusion process of Example 1.

| Protein | Buffer | Concentration (mg/ml Feed) |
|---|---|---|
| Collagen | PBS | 0.3 |
| Elastin | PBS | 0.3 |
| Chitosan | 99% PBS | 3.5 |
| | 1% Acetic Acid | |
| Hyaluronan | PBS | 0.5 |
| Chondroitin sulphate | PBS | 0.5 |

Referring to Table 1, the polymer solution that was utilized in this example consisted of proteins that were dissolved in physiological buffers based on phosphate buffered saline (PBS) with specific concentrations. In this example, the polymer solution includes 0.3 mg/ml of collagen, 0.3 mg/ml of elastin, 3.5 mg/ml of chitosan, 0.5 mg/ml of hyaluronan, and 0.5 mg/ml of chondroitin sulphate. Marine-source-derived pharmaceutical-grade chitosan (MW 100-250 kD) suitable for oral and systemic administration, solution of bovine type I atelocollagen solution suitable for medical device manufacture, pharmaceutical-grade sodium hyaluronate, elastin-soluble, and chondroitin sulphate derived from Porcine cartilage were utilized in this example.

Furthermore, five different scaffolds were prepared using five different polymer solutions as feed, namely, pure elastin, pure hyaluronan, pure collagen, pure chitosan, and the polymer solution with a composition set forth in Table 1. These polymer solutions included proteins and polysaccharides with a concentration of 500 μg/ml and were extruded through AAO nanopores that had a diameter of 140 nm.

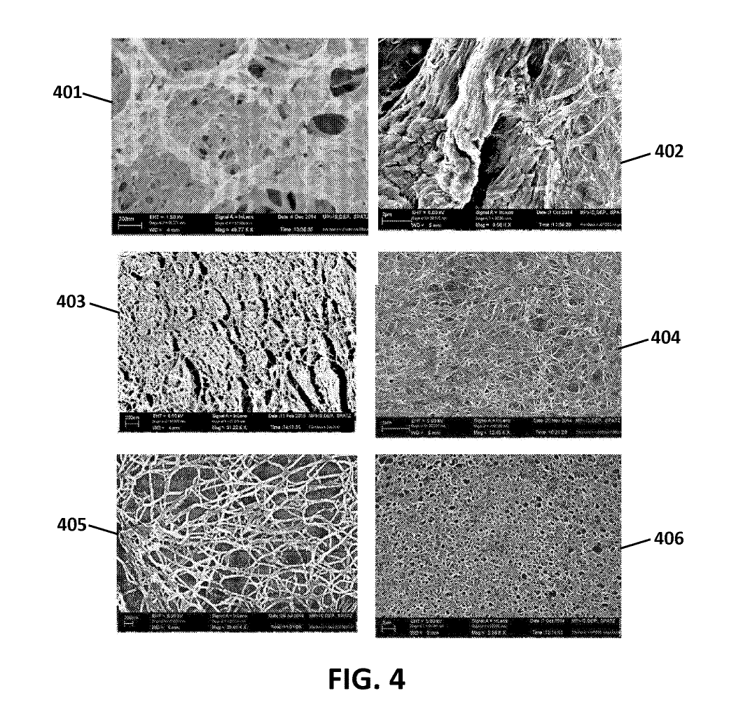

The produced nanofibrous scaffolds were characterized by scanning electron microscopy (SEM), transmission electron microscopy (TEM), atomic force microscopy (AFM), and confocal laser scanning microscopy. FIG. 4 illustrates SEM images of the produced nanofibrous scaffolds. A SEM image of the nanofibrous scaffold produced from the elastin polymer solution is designated by 401, a SEM image of the nanofibrous scaffold produced from the hyaluronan polymer solution is designated by 402, a SEM image of the nanofibrous scaffold produced from the collagen polymer solution is designated by 403, a SEM image of the nanofibrous scaffold produced from the chitosan polymer solution is designated by 404, a SEM image of a one-layer nanofibrous mat produced from the polymer solution of Table 1 is designated by 405, and a SEM image of a multi-layer nanofibrous scaffold produced from the polymer solution of Table 1 is designated by 406.

Referring to FIG. 4, the extruded nanofibers from the elastin polymer solutions have diameters of about 50 nm while extruded composite nanofibers from the polymer solution of Table 1 have a diameter of about 70 nm.

### Cell Culturing

Cell growth was carried out without any growth factor on the produced nanofiber scaffold. Fibroblast cells were grown on a first side of the scaffold and Keratinocyte cells on a second, opposing side. The cells were maintained in, a Dulbecco's modified Eagle's medium (DMEM) supplemented with 2 ml of RPMI-1640 with 10% fetal bovine serum (PBS) at 37° C. and 5% CO₂. The cells were seeded without any growth factor with a number of 5×10⁵ cells on the surface of the produced composite polymeric nanofibers in DMEM containing 1% FBS. Before seeding the cells on the prepared scaffold, cells were trypsinized with trypsin-EDTA 2.5% solution for 3 minutes. Cells were seeded at a density of 5×10⁵ per substrate in DMEM containing 1% FBS.

To explore the cell attachment and biocompatibility of the final product, i.e., nanofibrous scaffold seeded with cells, the growth of keratinocyte and fibroblast cells on nanofibrous scaffolds were studied. Furthermore, the nanofibers with an average diameter of 70 nm were deposited on glass slides as scaffold and cells were seeded on them, pursuant to the teachings of the present disclosure.

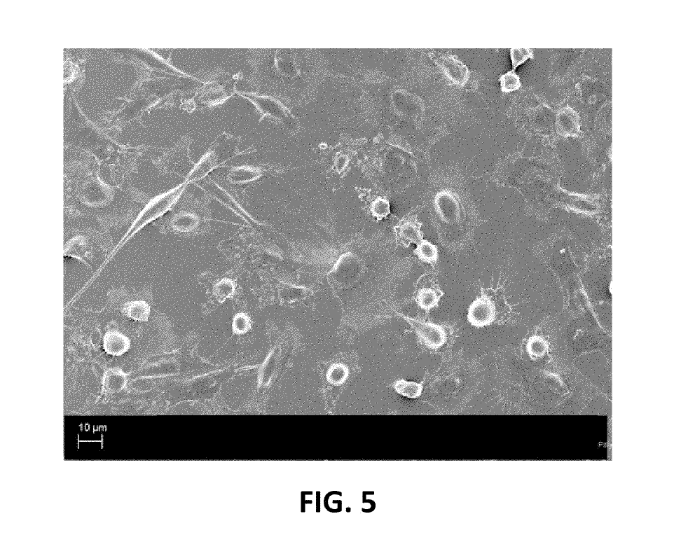

FIG. 5 illustrates a SEM image of the cultured cells on the composite nanofibrous scaffold. As shown in this figure, the cells have attached to the nanofibrous scaffold. Cell-seeded scaffolds prepared as described in Example 1 may be useful as a wound dressing or as a skin substitute configured to promote wound healing and/or tissue regeneration. In addition, the cell-seeded scaffolds can be used as an application for treatment of skin damage, including diabetic ulcers, injuries, wounds, or other dermatological conditions. Thus, a wound dressing or alternatively an artificial skin prepared pursuant to the teachings of the present invention may include the composite nanofibrous scaffold, where a layer of at least one type of cells is grown on at least one side of the scaffold.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated, immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that zany of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method of preparing a skin, regeneration scaffold, the method comprising:
preparing a polymer solution by dissolving, a biopolymer in a solvent;
subjecting the polymer solution to a template-assisted extrusion process with a nanoporous material as the template in order to produce polymer nanofibers; and
fabricating a multilayer composite nanofibrous scaffold using the polymer nanofibers.

2. The method according to claim 1, further comprising separating the polymer nanofibers from the solvent.

3. The method according to claim 1, further comprising subjecting the multilayer composite nanofibrous scaffold to a plastic compression.

4. The method according to claim 1, further comprising seeding the composite nanofibrous scaffold with cells, wherein the cells are selected from the group consisting of autologous cells, allogeneic cells, and combinations thereof.

5. The method according to claim 4, wherein the cells are selected from the group consisting of keratinocyte, fibroblasts, and combinations thereof.

6. The method according to claim 4, wherein the cells are selected from the group consisting of keratinocytes, fibroblasts, melanocytes, endothelial cells, chondrocytes, osteocytes, osteoblasts, stem cells, and bone marrow.

7. The method according to claim 4, wherein seeding the composite nanofibrous scaffold with the cells includes: growing a layer of a first type of cell on a first side of the scaffold and growing a second type of cell on a second side of the scaffold.

8. The method according to claim 7, wherein the first type of cell includes keratinocytes.

9. The method according to claim 7, wherein the first type of cell includes fibroblasts.

10. The method according to claim 7, wherein the first type of cell and the second type of cell are selected from the group consisting of keratinocytes, fibroblasts, melanocytes, endothelial cells, chondrocytes, osteocytes, osteoblasts, stem cells, and bone marrow.

11. The method according to claim 1, wherein the nanoporous material is selected from the group consisting of anodic aluminum oxide (AAO), titanium dioxide, silicon dioxide, polycarbonate, and a zeolite.

12. The method according to claim 1, wherein the nanoporous material has a mean pore size in a range of 4 nm to 900 nm.

13. The method according to claim 1, wherein the nanoporous material has a thickness in a range of 10 μm to 400 μm.

14. The method according to claim 1, wherein the nanoporous material is an AAO membrane with a mean pore size in a range of 10 nm to 150 nm.

15. The method according to claim 1, wherein the biopolymer is selected from the group consisting of proteins, polysaccharides, and combinations thereof.

16. The method according to claim 1, wherein the biopolymer is selected from the group consisting of fibronectin, elastin, fibrinogen, collagen, myosin, actin, BSA, α-actinin, laminin, chondroitin sulfate, hyaluronan, chitin-derivatives, and mixtures thereof.

17. The method according to claim 1, wherein the template-assisted extrusion process includes extruding the polymer solution through pores of the nanoporous material.

18. The method according to claim 17, wherein extruding the polymer solution through, pores of the nanoporous material is carried out by a method selected from the group consisting of pressing the polymer solution through the pores of the nanoporous material and drawing the polymer solution through the pores of the nanoporous material.

19. The method according to claim 1, wherein fabricating the multilayer composite nanofibrous scaffold using the polymer nanofibers includes depositing the polymer nanofibers in a layer-by-layer approach on a substrate.

20. The method according to claim 1, further comprising applying mechanical pressure on the multilayer composite nanofibrous scaffold by a plastic compressor.

21. The method according to claim 1, further comprising cross-linking the multilayer composite nanofibrous scaffold by a freezing-thawing method.

22. The method according to claim 21, wherein the freezing-thawing method includes freezing the scaffold at −20° C. and thawing the scaffold to room temperature or wherein the plastic compressor method the scaffold get under special force (depends on size of scaffold) on special temperature.

23. A skin regeneration scaffold prepared by the method of claim 1.

24. A skin substitute, the skin substitute comprising a multilayer composite nanofibrous scaffold seeded with keratinocytes and fibroblasts cells.
